(19) United States
(12) Patent Application Publication
(10) Pub. No.: US 2014/0323014 A1
(43) Pub. Date: Oct. 30, 2014

(54) Method For Extracting Drone Bee Larvae With Maximum Biological Value

(71) Applicants: Vyacheslav Nikolaevich Trifonov, Zarechni (RU); Julia Anatoljevna Elistratova, Penza (RU); Konstantin Gennadievich Elistratov, Penza (RU); Natalia Vyacheslavovna Kurus, Penza (RU); Tatiana Viktorovna Elistratova, Penza (RU); Lilia Alexandrovna Burmistrova, Hodynino (RU); Natalya Valentinovna Budnikova, Hodynino (RU)

(72) Inventors: Vyacheslav Nikolaevich Trifonov, Zarechni (RU); Julia Anatoljevna Elistratova, Penza (RU); Konstantin Gennadievich Elistratov, Penza (RU); Natalia Vyacheslavovna Kurus, Penza (RU); Tatiana Viktorovna Elistratova, Penza (RU); Lilia Alexandrovna Burmistrova, Hodynino (RU); Natalya Valentinovna Budnikova, Hodynino (RU)

(21) Appl. No.: 14/360,718

(22) PCT Filed: Jul. 5, 2012
(86) PCT No.: PCT/RU12/00541

§ 371 Date: May 27, 2014

(30) Foreign Application Priority Data
Dec. 29, 2011 (RU) 2011153905

(51) Int. Cl. A23L 1/076; A61K 35/64; A23L 1/30

(52) U.S. Cl. CPC A23L 1/076; A23L 1/30; A61K 35/642
USPC 449/1

(57) ABSTRACT

The proposed method relates to the pharmaceutical and foods industries and to medicine, in particular to biologically active dietary supplements (BADS), and describes a process for extracting drone bee larvae for producing a drone bee brood with maximum biological value.

### CROSS-REFERENCE TO RELATED APPLICATIONS

This application is a U.S. national stage application of an international application PCT/RU2012/000541 filed on 5 Jul. 2012, published as WO/2013/100802, which international application claims priority of a Russian Federation patent application RU2011153905 filed on 29 Dec. 2011.

### FIELD OF THE INVENTION

The invention relates to the pharmaceutical, food industry, and medicine, specifically to biologically active dietary supplements (BADS) to food, and describes a process of selection of drone larvae for obtainment of the drone brood.

### BACKGROUND OF THE INVENTION

In Romania, it was patented a method of obtainment of a biologically active product from larvae of drones or working bees or queens (Patent RO No. 74872/1980), that is a biologically active substance for obtainment of medicines and cosmetic means. There is known a Russian Federation Patent RU No. 2402920 “Method of preparation of the fodder supplement from drone larvae for increase of dogs organisms resistance by parasitosis”. There is known a Russian Federation Patent RU No. 2258522 “Method of preparation making from drone larvae for stimulation of animal organism”. There is known a Russian Federation Patent RU No. 2395289 “Method of making of biogenic stimulator from the larvae of drone brood of bees”. There is known a Russian Federation Patent RU No. 2245155 “COMPOSITION OF THE CANNED HOMOGENATE OF THE BEES BROOD (DRONE BROOD AND QUEEN LARVAE)”.

In the Romanian patent RO No. 74872/1980, it is disclosed that larvae are collected before sealing of cells with larvae at 10-th day after eggs laying. In Patent RU No. 2258522 it is said: the preparation is made from the drone larvae of each age. In Patent RU No. 2402920: it is made collection of larvae 9-10 days old. In Patent RU No. 2395289: the age of drone larvae: 18-22 days. During numerous works by definition of biological activity of drone brood we have established the term of the drone larvae selection for processing.

Bee queen lays unfertilized eggs, from which drones develop. The period of embryonic development (the stage of egg) forms 3-3.5 days. By larvae withdrawal from the eggs, post-embryonic development begins that is accompanied by complete metamorphosis with passing of the stages of larva, prepupa, pupa and imago. Metamorphosis is regulated by hormones. The stage of larva (near 7 days) is characterized by intensive nutrition. From the 7-th to 10.5-th days (the counting is made from the zero day) the larvae of drones finish nutrition, straighten, settling by the head end to the aperture of cell, which is sealed by bees with porous wax lid, the stage of prepupa begins (from 10-th to 14-th day). In prepupae fat body is well developed, organs of vision, sense of smell, pigmentation are absent. During all the larval stage formation of testicles proceeds, at the end of which testicles reach size of the adult insect. From 15-th to 24-th days (from the moment of the egg laying)—stage of pupa, which externally resembles adult drone. After termination of the stage of pupa the adult drone quits the cell and comes out on the surface of a honeycomb.

Temporal interval in each age period is explained by the force of the bees family and its provision by the food. For this reason it is impossible to provide the instruction for the bee keeper, that it is necessary to collect larvae just in the given day, since biological value of a drone brood retrieved will be different in different families of bees.

### SUMMARY OF THE INVENTION

The authors of the present invention have discovered an external feature of drone brood selection: prepupae in the sealed cells, occupying vertical position with absence of pigmentation or pupae with colorless or slightly yellowing eyelets. It is undesirable using pupae with the expressed pigmentation (eyelets with violet tint, skin covers with yellowish one) in the processing.

Prepupae, pupae with yellowing eyelets have highest contents of prohormones. The aforementioned feature is highly technological, because it permits a visual selection of necessary pupae and avoiding costly research of biological activity of the collected drone brood. The aforementioned feature permits standardizing the collected drone brood by acting substances.

### PREFERRED EMBODIMENTS OF THE INVENTION

### Example (1)

If immature larvae are collected, i. e. without the visual selection utilizing the aforementioned feature, then the larvae are situated not vertically, but are twisted as a worm. Immature larvae provide a lesser amount of decenoic acids.

### Example (2)

The larvae with blue eyelets contain excrements, and also a low content of hormones, such as: estradiol, prolactin, and progesterone.

Composition and properties of the drone brood, extracted by the inventive method, also have differences. Thus, the contents of basic sexual hormones (testosterone, progesterone, prolactin, esradiol) in 100 g of the drone brood, produced by the method claimed, determined by radio-immune methods with the use of standard sets “Immunotech” (Czech Respublic) on radioisotopic apparatuses was maximal (see Table 1). By extracting of drone brood according to the method claimed, there are reached maximal contents of unsaturated compounds (by the lowermost index of oxidability), and decenoic acids (Table 2). By extracting of drone brood by the other method (i.e. by the example 1), the mentioned parameters were lower.

TABLE 1
Contents of testosterone, progesterone, prolactin and estradiol in drone
brood by the method claimed and by another method

| | Drone brood by | Drone brood by the method |
|---|---|---|
| Indexes | the example 1 | claimed |
| Testosterone, nmol/100 g | Less than 0.292 | From 0.292 to 0.322 |
| Progesterone, nmol/100 g | Less than 42.36 | From 42.63 to 60.01 |
| Prolactin, nmol/100 g | Less than 344.6 | From 344.6 to 475.4 |
| Estradiol, nmol/100 g | Less than 431.2 | From 431.2 to 847.9 |

TABLE 2
Composition and properties of the drone brood, obtained
by the method claimed

| | Drone brood by | Drone brood by the |
|---|---|---|
| Indexes | the example 1 | method claimed |
| Contain of unsaturated | More than 10.0 | Less than 10.0 |
| compounds (by index of | | |
| oxidation, c) | | |
| Mass part of decenoic acids, % | Less than 3.0 | More than 3.0 |
| Mass part of raw protein | Less than 26% | From 28.0 to 32.0 |
| Concentration of hydrogen | Less than 6.1 | From 6.1 to 6.8 |
| ions (pH) | | |

Therefore, the inventive method of selection of drone larvae for preparation of drone brood includes the step of:

extracting prepupae from sealed cells; said prepupae occupy a vertical position, and said prepupae are free of pigmentation; or

extracting pupae from sealed cells with colorless or yellowish eyes;

wherein said method results in obtaining the following characteristics of the drone brood:

(1) Testosterone no less than 0.292 nmol/100 g;

(2) Progesterone no less than 42.63 nmol/100 g;

(3) Prolactin no less than 344.6 nmol/100 g;

(4) Estradiol no less than 431.2 nmol/100 g;

(5) Unsaturated compounds, indexes of oxidability less than 10.0 c;

(6) Decenoic acids no less than 3.0%;

(7) Raw protein from 28.0 to 32.0%; and

(8) Concentration of hydrogen ions (pH) from 6.1 to 6.8.

1. A method of selection of drone larvae for preparation of drone brood, including:
extracting of prepupae, occupying vertical position, with absence of pigmentation or pupae with colorless or slightly yellowing eyes from the cell sealed, permitting to reach the contain of testosterone, progesterone, prolactin and estradiol in the drone brood with characteristics:
(1) Testosterone no less than 0.292 nmol/100 g;
(2) Progesterone no less than 42.63 nmol/100 g;
(3) Prolactin no less than 344.6 nmol/100 g;
(4) Estradiol no less than 431.2 nmol/100 g;
(5) Unsaturated compounds, indexes of oxidability less than 10.0 c;
(6) Decenoic acids no less than 3.0%;
(7) Raw protein from 28.0 to 32.0%;
(8) Concentration of hydrogen ions (pH) from 6.1 to 6.8.

2. A method of selection of drone larvae for preparation of drone brood, said method including the steps of:
extracting prepupae from sealed cells; said prepupae occupy a vertical position, and said prepupae are free of pigmentation; or
extracting pupae from sealed cells with colorless or yellowish eyes.
